(19) United States
(12) Patent Application Publication
(10) Pub. No.: US 2021/0052763 A1
(43) Pub. Date: Feb. 25, 2021

(54) COMPACT DUAL-INTAKE DIFFUSER

(71) Applicant: BELIEVE IT LLC, Savannah, NY (US)

(72) Inventor: Jiaxin Guo, Shenzhen (CN)

(21) Appl. No.: 16/546,036

(22) Filed: Aug. 20, 2019

(51) Int. Cl. A61L 9/12; A61L 9/14

(52) U.S. Cl. CPC A61L 9/127; A61L 2209/132; A61L 9/14

(57) ABSTRACT

A dual-intake diffuser that has a housing, a selectively attachable base, a selectively attachable cap, a wick, and a diffuser element. The selectively attachable base and selectively attachable cap are configured to receive a composition. The diffuser element is configured to diffuse liquid at an opening in the cap. The diffuser system may include a connector and lighting element.

### BACKGROUND

### Technical Field

This disclosure generally relates to diffusers. More specifically, the present disclosure relates to compact dual-intake diffusers.

### Related Technology

Diffusers may be used for aromatherapy. Aromatherapy is the practice of using the natural oils extracted from flowers, bark, stems, leaves, roots or other parts of a plant to enhance an individual's psychological and physical well-being. Aromatherapy, as indicated by its name, enhances psychological and physical well-being through smell.

Many practitioners of this art routinely employ a variety of natural oil extracts to enhance or otherwise affect either their own or another person's physiological and physical well-being. Oils, combinations of oils, and combinations of oils with water or other material can be used to decrease stress, increase productivity, or promote clarity of thought. In some circumstances, the oils are applied directly to the skin of the person who desires their beneficial effects. In other instances, a diffuser is used to diffuse the oils into the environment surrounding the person who desires their beneficial effects. Thus, diffusers can enhance an individual's psychological and physical well-being.

While there are many types of diffusers on the market, pour-top diffusers provide certainty. Those who are sick or otherwise afflicted may use essential oils and other diffused liquids. In some cases, the sick individual may desire a certain amount of oil to be diffused but may not be well enough to operate the diffuser him or herself. In such an instance a supporting family member or friend may start the diffuser on the sick individual's behalf. However, the family member or friend may not have the time, energy, etc., to stay by the sick individual's side throughout diffusion. In such an instance, pour-top diffusers allow the supporting family member or friend to pour the precise amount of oil to be diffused into the diffuser prior to diffusion. In this way, the sick individual does not have to be concerned that more than the desired amount of liquid will be diffused if the supporting family member or friend is no longer nearby to turn off the diffuser at the appropriate time. Pour-top diffusers also facilitate creation of essential oil blends, concentrations of essential oils, etc. The user can pour the exact amount of each ingredient needed and know with certainty that no additional liquid will be diffused.

Bottom-collection diffusers provide ongoing diffusion. A homeowner preparing for a party may desire constant diffusion throughout the gathering to elevate the mood, provide a certain ambiance, etc. If using a pour-top diffuser, it can be a hassle for the homeowner to return to the diffuser periodically to add additional oil, water, etc. Bottom-collection diffusers avoid this problem by continually absorbing available liquid. If a constant, or large enough, supply of liquid is provided, a bottom-collection diffuser will provide diffusion continually throughout the entire event. Likewise if the user has a large amount of liquid and wants to diffuse all of it without having to refill the diffuser. In addition, there are many other diffusion applications for which ongoing diffusion is preferable, such as parents taking care of young children, work gatherings, etc.

One potential drawback with diffusers is their lack of portability. It may be difficult for the user to find room in her car, suitcase, etc., to fit a diffuser that is too large Likewise, many diffusers are powered through a standard AC outlet. If the user is traveling and does not have access to a standard AC outlet she may not be able to use her diffuser even though her medical regimen may call for its use. This problem of portability is greatly exacerbated if the user needs to use two types of diffusers for different applications. In that instance, the user must pack both diffusers and any related accessories.

It is difficult to combine the certainty of pour-top diffusers with the ongoing diffusion of bottom-collection diffusers, as each requires its own mechanical setup and doing so results in a large, cumbersome diffuser that is not easily portable and demands a large amount of storage space.

Accordingly, there are many disadvantages with diffusers that can be addressed.

### BRIEF SUMMARY

Implementations of the present disclosure solve one or more of the foregoing or other problems in the art with systems, methods, and apparatuses for a compact dual-intake diffuser. In particular, one or more implementations can include a diffuser system having a housing with at least two ends and a selectively attachable base that is selectively attachable to at least one end of the housing and that is configured to receive a composition. The diffuser system further may have a selectively attachable cap that is selectively attachable to at least one end of the housing and that has at least one opening. Finally, the diffuser system may have a diffusing element that is configured to diffuse the composition at the opening of the selectively attachable cap.

Diffuser systems of the present disclosure may further include a connector for communicating with a power source, a wick in association with the selectively attachable base and selectively attachable cap, a spring in association with the selectively attachable base and selectively attachable cap, and a diffuser element.

Accordingly, a compact dual-intake diffuser is disclosed.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope.

In the drawings, multiple instances of an element may each include separate letters appended to the element number. For example, two instances of a particular element “100” may be labeled as “100a” and “100b.” In that case, the element label may be used without an appended letter (e.g., “100”) to generally refer to every instance of the element, while the element label will include an appended letter (e.g., “100a”) to refer to a specific instance of the element. Similarly, a drawing number may include separate letters appended thereto. For example, FIG. 2 may include FIG. 2A and FIG. 2B. In that case, the drawing number may be used without the appended letter (e.g., FIG. 2) to generally refer to every instance of the drawing, while the drawing label will include an appended letter (e.g., FIG. 2A) to refer to a specific instance of the drawing. The disclosure will be described and explained with additional specificity and detail through the use of the accompanying drawings in which:

FIG. 1 illustrates a front perspective view of a compact dual-intake diffuser according to the present disclosure.

FIG. 2 illustrates an exploded front perspective view of a portion of a compact dual-intake diffuser according to the present disclosure.

FIG. 3 illustrates the compact dual-intake diffuser of the present invention placed in a wine glass with liquid to be diffused.

FIG. 4 is an exploded perspective view of a compact dual-intake diffuser of the present disclosure.

FIG. 5 is a zoomed in cross sectional view of a portion of a compact dual-intake diffuser of the present disclosure.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Unless defined otherwise, all technical and scientific terms used herein have the same meaning as commonly understood by one of ordinary skill in the art to which the present disclosure pertains.

Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

It will be noted that, as used in this specification and the appended claims, the singular forms “a,” “an” and “the” include plural referents unless the context clearly dictates otherwise. Thus, for example, reference to a “salt formation” includes one, two, or more salt formations. Similarly, reference to a plurality of referents should be interpreted as comprising a single referent and/or a plurality of referents unless the content and/or context clearly dictate otherwise. Thus, reference to “salt formations” does not necessarily require a plurality of such salt formations. Instead, it will be appreciated that independent of conjugation; one or more salt formations are contemplated herein.

As used herein, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure and/or claimed invention.

Various aspects of the present disclosure can be illustrated by describing components that are bound, coupled, attached, connected, and/or joined together. As used herein, the terms “bound,” “coupled”, “attached”, “connected,” and/or “joined” are used to indicate either a direct association between two components or, where appropriate, an indirect association with one another through intervening or intermediate components. In contrast, when a component is referred to as being “directly bound,” “directly coupled”, “directly attached”, “directly connected,” and/or “directly joined” to another component, no intervening elements are present or contemplated. Furthermore, binding, coupling, attaching, connecting, and/or joining can comprise mechanical and/or chemical association.

To facilitate understanding, like reference numerals (i.e., like numbering of components and/or elements) have been used, where possible, to designate like elements common to the figures. Specifically, in the exemplary embodiments illustrated in the figures, like structures, or structures with like functions, will be provided with similar reference designations, where possible. Specific language will be used herein to describe the exemplary embodiments. Nevertheless, it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

Any headings and subheadings used herein are for organizational purposes only and are not meant to be used to limit the scope of the description or the claims.

Although the subject matter described herein is provided in language specific to structural features and/or methodological acts, it is to be understood that the subject matter defined in the appended claims is not necessarily limited to the described features or acts so described. Rather, the described features and acts are disclosed as example forms of implementing the claims.

### Overview of Compact Dual-Intake Diffusers

As briefly discussed above, pour-top and bottom-collection diffusers each serve distinct purposes. Pour-top diffusers provide certainty because the user can determine the exact amount of essential oil to diffuse and may leave the diffuser unattended without risk that additional oil will be diffused. Bottom-collection diffusers, on the other hand, facilitate ongoing, hassle free diffusion. Enjoying the benefits of both pour-top and bottom-collection diffusers, especially in a portable setup, is difficult.

Embodiments of the present disclosure enable the portable diffusion of essential oils. A compact dual-intake diffuser as described herein may include top and bottom openings for the provision of essential oils, facilitating pour-top and bottom-collection intake for diffusion. The compact dual intake diffuser described herein solves one or more problems in the art of diffusers. Advantageously, the diffuser combines pour-top and bottom-collection diffusion properties into one single diffuser, while being compact for ease of storage and increased portability.

### Compact Dual-Intake Diffuser

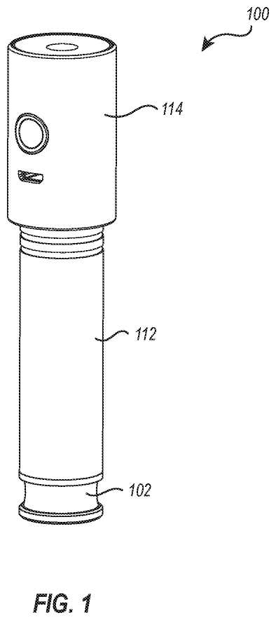

Reference will now be made to the figures of the present disclosure. For example, FIG. 1 illustrates a perspective view of a diffuser system according to one or more implementations of the present disclosure. The diffuser 100 may comprises a base 102, housing 112, cap 114, and diffusing element 120.

The base, as depicted in FIG. 1, may be circular in shape, having a flat bottom surface. However, it should be understood that base 102 may be any shape, such as spherical, ovoid, prismatic, oblong, polygonal, or any combination and/or variation of the foregoing. In certain embodiments the circular shape of base 102 may provide the benefits of being ergonomic and/or easy to handle. In addition, the flat bottom surface of base 102 may promote the stability of the diffuser 100 when in a prone position and may additionally encourage anything placed within the diffuser 100 (e.g., the diffusible composition) to rest within the diffuser 100 and not leak out of the diffuser 100 (e.g., through an unobstructed first opening).

As noted previously, it should be appreciated that the base 102 and/or contouring of the diffuser is not limited to what is depicted in the accompanying figures. Rather, the diffuser 100 may be of any shape and may include any contour. For example, the diffuser 100 may be spherical, ovoid, prismatic, oblong, polygonal, irregular shape, or any combination and/or variation of the foregoing. Further, although the housing 112, cap 114, and diffusing element 120 are depicted as being substantially circular, the housing 112, cap 114, and diffusing element 120 may be of any shape and of any size. Base 102, housing 112, and cap 114 may comprise any suitable material, including without limitation plastics, thermoplastics, thermoplastic elastomers, elastomers, rubbers, bamboo, woods, glass, or combinations thereof.

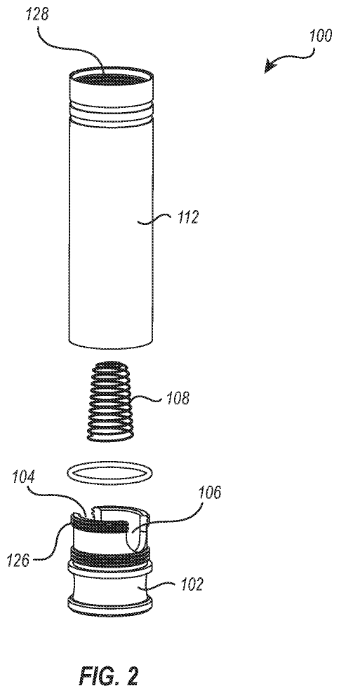

As shown in FIG. 2, the base 102 may screw into an end of housing 112 by means of threads 126. As is well known in the art, the helical properties of threads 126 allow base 102 to remain attached to housing 112 even if base 102 is not fully screwed onto housing 112. Such an arrangement is particularly useful for bottom-collection diffusion. Base 102 may be partially screwed onto base 102 and the entire diffuser 100 placed in a glass or other liquid container. Base 102 may have a first opening 104 and second opening 106 configured to receive a composition.

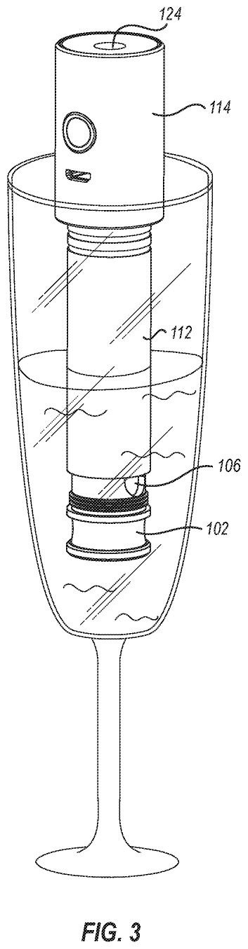

For example, as shown in FIG. 3, diffuser 100 may be placed in a wine glass or other liquid container such that standing liquid in the bottom of the container covers first opening 104 and second opening 106 when base 102 is only partially screwed onto housing 112. Alternatively, a fluid source designed to provide a composition may connect to base 102 to provide a composition that enters through first opening 104 and second opening 106. When diffuser system 100 is placed in a glass or other liquid container, first opening 104 and second opening 106 may receive liquid such that the liquid meets wick 110 and is then wick drawn to diffuser element 122 for diffusion through aperture 124.

Alternatively, the user may fully screw base 102 onto housing 112 for purposes of pour-top diffusion. In such an arrangement, with the base 102 fully screwed onto housing 112, the user may unscrew cap 114 from housing 112, and pour liquid into the aperture 128 of housing 112. With base 102 fully screwed onto housing 112, the liquid that is poured through aperture 128 of housing 112 is retained in base 102, meets wick 110, and may be wick drawn to diffuser element 122 for diffusion through aperture 124.

As stated, base 102 may have first opening 104 and second opening 106. In some embodiments, first opening 104 and second opening 106 may only be visible when base 102 is partially or completely unscrewed from housing 112. In some embodiments, first opening 104 and second opening 106 may be visible regardless of whether base 102 is unscrewed from housing 112.

Referring to FIG. 2, first opening 104 and second opening 106 may in some embodiments be u-shaped. However, it should be appreciated that the shape of first opening 104 and second opening 106 is not limited to what is depicted in the accompanying figures. Rather, first opening 104 and second opening 106 may be of any shape and may include any contour. For example, first opening 104 and second opening 106 may be spherical, ovoid, prismatic, oblong, polygonal, or any combination and/or variation of the foregoing.

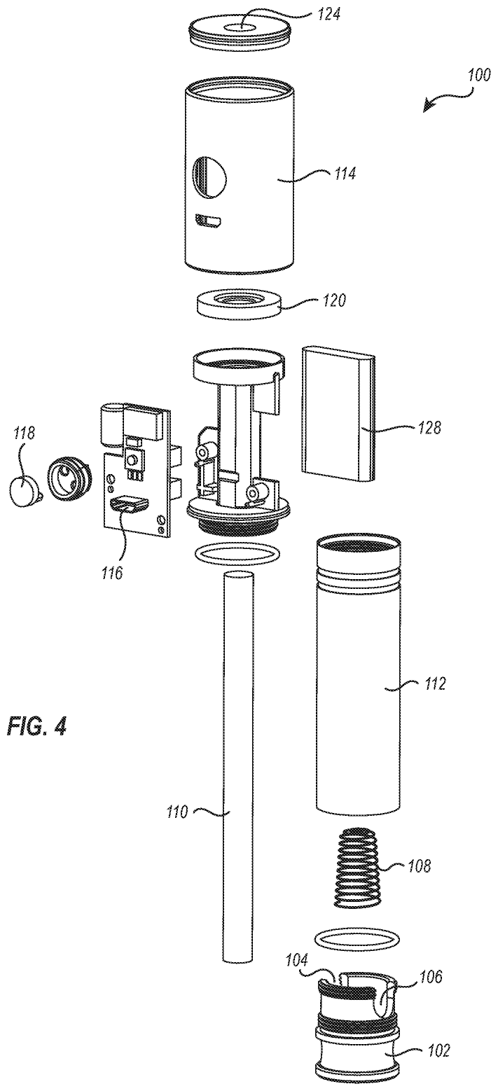

Referring again to FIG. 2 and also to FIG. 4, in some embodiments base 102 may include a spring 108. Spring 108 may horizontally and vertically stabilize wick 110. Such horizontal and vertical stabilization of wick 110 may increase the diffusion efficiency of diffuser 100 by ensuring wick 110 remains in contact with spring 108 and diffusing element 120 as often as possible. In some embodiments, spring 108 may be sized and configured to receive an end of wick 110 and hold it in alignment. Likewise, in some embodiments, diffusing element 120 may be sized and configured to receive an end of wick 110 and hold it in alignment.

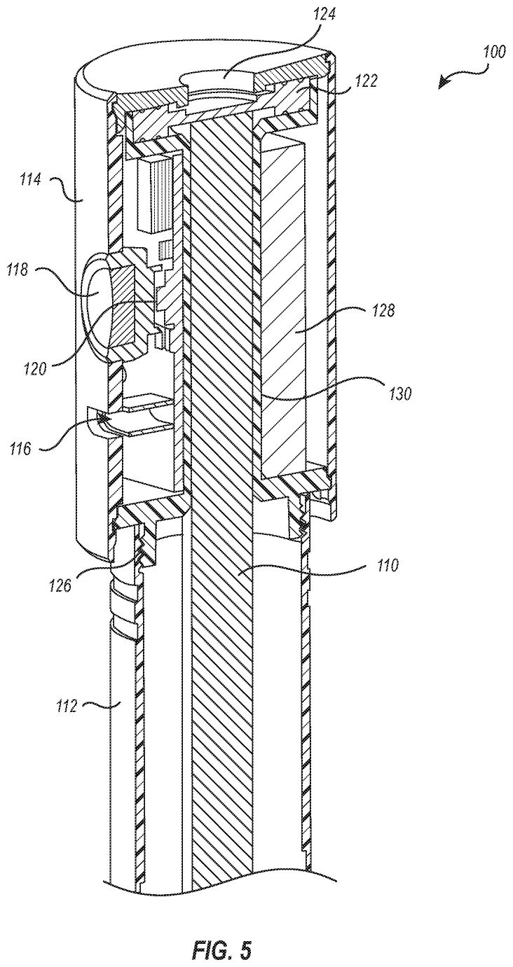

Referring now to FIG. 5, the diffuser 100 may include a cap 114. The cap 114 may have a connector 116, button 118, lighting element 120, diffusing element 122, and aperture 124.

Connector 116 may connect to and/or draw power from a power source, such as, for example, an AC power cord having a complementary connector port. As a non-limiting example, the connector 116 associated with the diffuser 100 may connect through an AC power cord to a wall jack in a home or office and draw power from the home or office to run one or more elements of the diffuser 100 (e.g., the diffusing element 122). The connector may be any connector that is configured to connect the diffuser 100 to a power source having sufficient power to run one or more elements of the diffuser. In addition to the power source of a home or business, exemplary power sources include a laptop computer, a desktop computer, a mobile phone, a digital music player, and similar electronic and/or computing devices.

Button 118 may turn the diffusion function of the diffuser 100 on and off. For example, if the diffuser 100 is not emitting diffused liquid, the user may push the button 118 to begin diffusion. Conversely, if the diffuser 100 is emitting diffused liquid, the user may push the button 118 to cease diffusion. In some embodiments, diffuser 100 may perform additional functions based on specific numbers of pushes of button 118 by the user. For example, if the user pushes button 118 twice in a row in rapid succession, diffuser 100 may diffuse liquid at twice the rate as compared to when the user pushes button 118 only once.

Lighting element 120 may be a single LED or a plurality of LEDs, such as one or more LED arrays. If a plurality of LEDs are used, they may be arranged in, for example, a line, a circle, a square, a flower shape, a rainbow shape, or any other desired shape or arrangement. Lighting element 120 may employ LEDs of the same or different colors. For example, red, white, and blue LEDs could be used during the Fourth of July holiday. One of skill in the art will understand that lighting element 120 may be of any height, shape, or material so as to light diffuser 100 from the interior thereof.

In one embodiment, lighting element 120 may provide sufficient light, i.e., is of a sufficient luminous intensity, to function as a nightlight. That is, lighting element 118 may have a luminous intensity of at least 1300 millicandela (mcd) at 20 milliamps (mA) or at least 5000 mcd at 20 mA. Alternatively, lighting element 120 may have a low nominal luminous intensity on the order of a few hundred mcd. Lighting element 120 may also use multiple LED arrays, each having a specific luminous intensity, either the same or different from each other. If lighting element 120 consists of multiple LED arrays they may be controlled independently or together, to provide an ornamental design.

As shown in FIG. 5, the diffuser 100 may include a diffusing element 122. The diffusing element 122 may include any element known in the art for diffusing compositions, including without limitation, a piezoelectric element, a heating element, a nebulizer, an ultrasonic atomizing sheet, or combinations thereof. Such diffusing elements are well known in the art, such as that described in U.S. Pat. Nos. 6,232,129, 3,840,758, 5,518,179, 4,432,699, and 5,622,897, each of which is incorporated herein by reference.

In some embodiments diffuser 100 may include an aperture 124 near the center of the top of cap 114 to release the diffused liquid. Aperture 124 may be of any size and shape that allows for the diffusion of liquid and is not limited to the size or shape shown in FIG. 5. Additionally, aperture 124 may be made of multiple smaller openings, rather than one large opening, which may be oriented in any number of arrangements.

Referring again to FIG. 5, a cross-section of the inside of housing 112 and cap 114 is shown. In some embodiments, housing 112 screws on to an inner member of cap 114 via threads 126 to ensure proper connection. Wick 110 fits snugly against diffusing element 122. This snug fit allows liquid that is poured in base 102 to be wick-drawn through wick 110 directly to diffusing element 122. Also shown is rechargeable battery 128 which can be of any suitable variety as known in the art. Rechargeable batteries are well known in the art, such as the rechargeable alkaline and nickel cadmium (NiCad) batteries discussed in U.S. Pat. Nos. 5,994,878 and 5,747,964, each of which is incorporated herein by reference. Rechargeable battery 128 may be insulated from wick 110 by wall 130. Wall 130 may comprise any suitable material, including without limitation, aluminum and stainless steel.

### Conclusion

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. An apparatus for diffusing a composition, the apparatus comprising:
a housing with at least two ends;
a selectively attachable base, wherein the selectively attachable base is selectively attachable to at least one end of the housing and is configured to receive a composition;
a selectively attachable cap with a diffusion opening, wherein the selectively attachable cap is selectively attachable to at least one end of the housing and is configured to receive a composition;
a wick; and
a diffusing element, wherein the diffusing element is configured to diffuse the composition at the opening of the selectively attachable cap.

2. The apparatus as in claim 1, wherein the selectively attachable base is circular.

3. The apparatus as in claim 1, wherein the selectively attachable base is threaded.

4. The apparatus as in claim 1, wherein the selectively attachable base further comprises a first and second opening configured to receive a composition.

5. The apparatus as in claim 2, wherein the first opening and second opening are u-shaped.

6. The apparatus as in claim 1, wherein the selectively attachable cap is threaded.

7. The apparatus as in claim 1, wherein at least a portion of the composition is diffused at the diffusing element after being wick-drawn.

8. The apparatus as in claim 1, further comprising a wick in association with the selectively attachable base and the selectively attachable cap.

9. The apparatus as in claim 5, wherein the wick is at least partially contained within the housing.

10. The apparatus as in claim 5, further comprising a spring in association with the wick and the selectively attachable base.

11. The apparatus as in claim 1, wherein the diffusing element comprises an ultrasonic atomizing sheet.

12. The apparatus as in claim 1, further comprising a connector for communicating with a power source.

13. The apparatus as in claim 1, wherein the selectively attachable base and the selectively attachable cap screw onto the housing.

14. The apparatus as in claim 1, further comprising a lighting element disposed at least partially within the cap.

15. The apparatus as in claim 1, further comprising a connector.

16. An apparatus for diffusing a composition, the apparatus comprising:
a housing with a proximal and distal end;
a selectively attachable base, wherein the selectively attachable base is selectively attachable to the proximal end of the housing and further comprises a first and second opening;
a selectively attachable cap with a diffusion opening, wherein the selectively attachable cap is selectively attachable to the distal end of the housing and is configured to receive a composition;
a wick; and
a diffusing element, wherein the diffusing element comprises an ultrasonic atomizing sheet and is configured to diffuse the composition at the opening of the selectively attachable cap.

17. The apparatus as in claim 16, further comprising a spring.

18. The apparatus as in claim 17, wherein the wick is in association with the spring and the diffusing element.

19. The apparatus as in claim 16, wherein the spring and the diffusing element hold the wick in horizontal and vertical alignment to facilitate diffusion.

20. The apparatus as in claim 16, further comprising a connector.
